(19) United States
(12) Patent Application Publication
(10) Pub. No.: US 2025/0210203 A1
(43) Pub. Date: Jun. 26, 2025

(54) APPARATUSES, COMPUTER-IMPLEMENTED METHODS, AND SYSTEMS FOR GENERATING AND DYNAMICALLY UPDATING A METRIC PROGRESS AGGREGATION INSIGHT INTERFACE IN ASSOCIATION WITH A PRE- OR POST-TRANSPLANT RECIPIENT

(71) Applicant: CareDx, Inc., Brisbane, CA (US)

(72) Inventor: Charlie NELSON, Brisbane, CA (US)

(21) Appl. No.: 19/074,798

(22) Filed: Mar. 10, 2025

Related U.S. Application Data
(63) Continuation of application No. 17/364,414, filed on Jun. 30, 2021.
(60) Provisional application No. 63/066,037, filed on Aug. 14, 2020.

(51) Int. Cl. G16H 50/30; G16H 10/60; G16H 50/70

(52) U.S. Cl. CPC G16H 50/30; G16H 10/60; G16H 50/70

(57) ABSTRACT

Methods, apparatuses, systems, and computer program products are disclosed for generating, outputting, and dynamically updating a metric progress aggregation insight interface in association with a pre- or post-transplant recipient. In an example embodiment, a computer-implemented method includes receiving, at disparate timing intervals and via one or more metric collection mechanisms, data related to a plurality of disparate metric collection inputs, accessing a metric targets profile, determining raw scores based on the metric targets profile, accessing a metric weights profile, determining, via relative importance determination logic, one or more aggregated scores based on the raw scores and the plurality of metric weights, generating a metric progress aggregation insight interface comprising the one or more aggregated scores, outputting the metric progress aggregation insight interface to at least a display of the portable electronic device, and dynamically updating the metric progress aggregation insight interface based on receiving additional data related to the plurality of disparate metric collection inputs.

### CROSS-REFERENCE TO RELATED APPLICATION

This application is a continuation of U.S. patent application Ser. No. 17/364,414, filed Jun. 30, 2021, which claims the benefit under 35 U.S.C. § 119(e) of U.S. Provisional Application No. 63/066,037, filed Aug. 14, 2020, the contents of each of which are incorporated herein by reference in their entirety for all purposes.

### FIELD OF THE DISCLOSURE

This disclosure relates generally to systems and methods for determining one or more scores representative of a plurality of activities and behaviors for a pre- or post-transplant recipient over a time window.

### BACKGROUND OF THE DISCLOSURE

Conventional systems and methods for tracking daily activities and behaviors suffer from deficiencies since they generally provide simplistic solutions for the average user who has no life-threating issues and mainly cares about improving physical health, body weight, etc. Therefore, conventional systems lack the capability to manage complex scenarios as is required in managing a transplant recipient's successful journey through an extended lifetime only made possibly by the provision of a donated organ or donated cells and only successful if the recipient is organized enough to maintain the donated organ or donated cells as best as possible. Staying organized is difficult given the amount of medications, medical visits, diagnostic tests that a transplant recipient must keep up with, and given the extraordinary circumstances that a transplant recipient faces day in, day out, dealing with constant threats of overmedication or undermedication (leading to dangers of opportunistic infections and malignant diseases when overmedicated or transplant rejection when undermedicated), and the daily reminder or fear that a rejection episode might mean the end of life. Furthermore, conventional systems and methods may not be capable of implementing dynamic changes needed to manage complex scenarios as described herein for the life of transplant recipients.

Furthermore, conventional systems and methods may not collectively factor in the multiple activities required in the life of a transplant recipient for determining whether a transplant recipient has met one or more metric targets. Additionally, the conventional systems and methods may not factor in a transplant recipient's activities over a given time window (e.g., multiple days, weeks, months). What is needed is a system capable of managing complex scenarios that takes into account multiple activities of the transplant recipient for one or more metric targets. What is also needed is a system that takes into account the transplant recipient's activities over a time window.

When in a particular situation, such of a user being in need of a transplanted organ or transplanted cells (pre-transplantation recipient or pre-allograft recipient), or a transplant recipient having already received a transplanted organ or transplanted cells (post-transplantation or post-allograft recipient) from another person, a comprehensive and unbiased approach to characterize how well the user or transplant recipient takes care of him or herself before and after transplantation is critically needed for the pre- or post-transplant recipient and the medical care provider. The present invention addresses this need.

### BRIEF SUMMARY OF THE DISCLOSURE

Disclosed herein is a system and associated methods for determining one or more scores representative of a pre- or post-transplant recipient's activities and behaviors for self-care to ensure that the transplant recipient is adequately cared for and the risk of avoidable allograft rejection is reduced. A pre- or post-transplant recipient may be a patient faced with late/end-stage organ disease (e.g., kidney, heart, lung, etc.) including a transplant patient, for example. Avoidable allograft rejection may arise when the transparent recipient lacks medication compliance by not taking his medication with the prescribed frequency or at the prescribed dosage.

The system and methods may include receiving the transplant recipient's input(s), measurement information, or both representative of a plurality of activities and behaviors for a transplant recipient. The system may determine one or more scores from this plurality of activities and behaviors. The transplant recipient's input(s) and/or measurement information may be related to one or more (health or behavioral) metrics and/or episodes, and the score(s) may be determined relative to one or more associated metrics or episodes. In some embodiments, the system and methods may use a numeric value to represent a metric. In some embodiments, the system and methods may use an indicator (e.g., a binary value, “completed” or “not completed,” “yes” or “no,” etc.) to represent an episode.

The score(s) may represent the transplant recipient's self-care status (e.g., how a patient is doing overall in taking care of himself or herself). The importance of a given metric to a transplant recipient's progress for self-care may vary depending on one or more factors such as the transplant recipient's health, age, gender, medical history, or the like, and one or more metric weights may be used to change the impact a metric has on the transplant recipient's score(s). The score(s) may be used by a pre- or post-transplant recipient to track overall health and well-being from home based on physical, physiological, and behavioral factors.

The system and associated methods disclosed herein may be used for pre-transplant selection of prospective transplant recipients and/or post-transplant care of transplant recipients to inform transplant recipients of his or her well-being, both from a physical, physiological, and behavioral point of view. The system and associated methods may help medical care providers identify and focus on the transplant recipients that require close attention, e.g., due to lack of physical and/or behavioral well-being. The information provided to the transplant recipient and/or medical care provider may be one or more scores. In some embodiments, a score may be a single numerical value. In some embodiments, the single score may be compared to an ideal score or scores of other transplant recipients within a certain cohort.

The score(s) may be used by a medical care provider to track the overall health and well-being of one or more pre- or post-transplant recipients. The score may be determined based on one or more physical, physiological, and/or behavioral factors. In some embodiments, the medical care provider may be able to track transplant recipients remotely. The system may identify one or more transplant recipients that may require extra attention, e.g., to adopt or reinforce habits necessary to reduce the risk of transplant rejection.

### BRIEF DESCRIPTION OF THE DRAWINGS

FIG. 1 illustrates an exemplary view of a user interface associated with a home screen of an app, according to embodiments of the disclosure.

FIG. 2 illustrates an exemplary view of a user interface associated with a tracking screen of an app, tracking one or more physical, physiological, and/or behavioral factors, according to embodiments of the disclosure.

FIG. 3 illustrates an exemplary flowchart for determining one or more pre- or post-transplant recipient scores, according to embodiments of the disclosure.

FIG. 4 illustrates an exemplary scoring time window, according to embodiments of the disclosure.

FIG. 5 illustrates an exemplary device that implements the above disclosed system, according to embodiments of the disclosure.

### DETAILED DESCRIPTION

Disclosed herein is a method for determining activities and behaviors of a pre- or post-transplant recipient to characterize how well the pre- or post-transplant recipient takes care of him or herself before and after transplantation. Also disclosed herein are computer-readable storage media and electronic devices for performing such methods.

In one aspect, disclosed herein is a method for determining activities and behaviors of a pre- or post-transplant recipient. The method may comprise: receiving data related to a plurality of metrics; accessing a metric targets profile, wherein the metric targets profile includes a plurality of metric targets related to the plurality of metrics; determining raw scores for the data related to the plurality of metrics, wherein the raw scores are determined based on the plurality of metric targets; accessing a metric weights profile, wherein the metric weights profile includes a plurality of metric weights related to the plurality of metrics; determining one or more scores based on the raw scores and the plurality of metric weights; and providing the one or more scores to the transplant recipient or a medical care provider, wherein the one or more scores are used to determine the activities and behaviors of the pre- or post-transplant recipient. Additionally or alternatively, in some embodiments, the receiving the data related to the plurality of metrics includes receiving input of the data. Additionally or alternatively, in some embodiments, the receiving the data related to the plurality of metrics includes taking one or more measurements using an electronic device, and wherein the providing the one or more scores includes displaying the one or more scores on the electronic device. Additionally or alternatively, in some embodiments, at least one of the plurality of metric targets is associated with an episode. Additionally or alternatively, in some embodiments, the method further comprises: changing the plurality of metric targets or the plurality of metric weights based on historical self-care status or a pre-set factor. Additionally or alternatively, in some embodiments, at least one of the raw scores may be a binary value. Additionally or alternatively, in some embodiments, the method further comprises: associating a bonus or a penalty with at least one of the plurality of metrics. Additionally or alternatively, in some embodiments, the one or more scores include a daily score. Additionally or alternatively, in some embodiments, the determining the one or more scores includes determining an overall score, the determining the overall score comprising: determining daily score weights for a plurality of daily scores within a scoring time window, wherein the overall score is based on the daily score weights and the plurality of daily scores. Additionally or alternatively, in some embodiments, the daily score weights vary and are based on a number of days since a current day. Additionally or alternatively, in some embodiments, the one or more scores provided to the pre- or post-transplant recipient are included in a personalized treatment regime for the pre- or post-transplant recipient, and the plurality of metric targets are based on an allogeneic organ transplant or allogeneic cells for the transplant recipient. Additionally or alternatively, in some embodiments, the recipient is a pre-transplant recipient. Additionally or alternatively, in some embodiments, the recipient is a post-transplant recipient. Additionally or alternatively, in some embodiments, one of the following: (a) the plurality of metrics is a plurality of health metrics and the plurality of metric targets is a plurality of health metric targets; (b) the plurality of metrics is a plurality of behavioral metrics and the plurality of metric targets is a plurality of behavioral metric targets; or (c) the plurality of metrics is a plurality of episodes and the plurality of metric targets is a plurality of episode targets. Additionally or alternatively, in some embodiments, the plurality of health metrics includes water consumption, calorie consumption, urination information, blood pressure, body weight, number of steps taken, number of hours slept, blood oxygen levels, blood glucose levels, or pulse. Additionally or alternatively, in some embodiments, the plurality of behavioral metrics includes medication adherence, mood levels, compliance with scheduled medical tests, compliance with medical office visits, or test results. Additionally or alternatively, in some embodiments, the plurality of episodes includes scheduling a blood or urine test, scheduling a medical office visit, attending a medical office visit, or receiving a blood or urine test.

In one aspect, a non-transitory computer-readable storage medium for determining activities and behaviors of a pre- or post-transplant recipient storing one or more programs is disclosed. The one or more programs comprises instructions, which when executed by one or more processors of an electronic device having display, cause the electronic device to: receive data related to a plurality of metrics; access a metric targets profile, wherein the metric targets profile includes a plurality of metric targets related to the plurality of metrics; determine raw scores for the data related to the plurality of metrics, wherein the raw scores are determined based on the plurality of metric targets; access a metric weights profile, wherein the metric weights profile includes a plurality of metric weights related to the plurality of metrics; determine one or more scores based on the raw scores and the plurality of metric weights; and provide the one or more scores to the pre- or post-transplant recipient or a medical care provider, wherein the one or more scores are used to determine activities and behaviors of the pre- or post-transplant recipient. Additionally or alternatively, in some embodiments, the electronic device receiving data related to the plurality of metrics includes taking one or more measurements using the electronic device, and wherein the electronic device providing the one or more scores includes displaying the one or more scores on the electronic device. Additionally or alternatively, in some embodiments, the determining the one or more scores includes determining an overall score, the determining the overall score comprising: determining daily score weights for a plurality of daily scores within a scoring time window, wherein the overall score is based on the daily score weights and the plurality of daily scores. Additionally or alternatively, in some embodiments, the daily score weights vary and are based on a number of days since a current day. Additionally or alternatively, in some embodiments, the plurality of metrics is a plurality of health metrics and the plurality of metric targets is a plurality of health metric targets, the plurality of health metrics includes water consumption, calorie consumption, urination information, blood pressure, body weight, number of steps taken, number of hours slept, blood oxygen levels, blood glucose levels, or pulse. Additionally or alternatively, in some embodiments, the plurality of metrics is a plurality of behavioral metrics and the plurality of metric targets is a plurality of behavioral metric targets, the plurality of behavioral metrics includes medication adherence, mood levels, compliance with scheduled medical tests, compliance with medical office visits, or test results. Additionally or alternatively, in some embodiments, the plurality of metrics is a plurality of episodes and the plurality of metric targets is a plurality of episode targets, the plurality of episodes includes scheduling a blood or urine test, scheduling a medical office visit, attending a medical office visit, or receiving a blood or urine test. Additionally or alternatively, in some embodiments, the one or more scores provided to the pre- or post-transplant recipient are included in a personalized treatment regime for the pre- or post-transplant recipient, and the plurality of metric targets are based on an allogeneic organ transplant or allogeneic cells for the transplant recipient. Additionally or alternatively, in some embodiments, the recipient is a pre-transplant recipient. Additionally or alternatively, in some embodiments, the recipient is a post-transplant recipient.

In one aspect, an electronic device for determining activities and behaviors of a pre- or post-transplant recipient is disclosed. The electronic device comprises: a display; one or more processors; memory; and one or more programs, wherein the one or more programs are stored in the memory and are configured to be executed by the one or more processors, the one or more programs including instructions for: receiving data related to a plurality of metrics; accessing a metric targets profile, wherein the metric targets profile includes a plurality of metric targets related to the plurality of metrics; determining raw scores for the data related to the plurality of metrics, wherein the raw scores are determined based on the plurality of metric targets; accessing a metric weights profile, wherein the metric weights profile includes a plurality of metric weights related to the plurality of metrics; determining one or more scores based on the raw scores and the plurality of metric weights; and displaying the one or more scores to the pre- or post-transplant recipient or a medical care provider, wherein the one or more scores are used to determine activities and behaviors of the pre- or post-transplant recipient. Additionally or alternatively, in some embodiments, the recipient is a pre-transplant recipient. Additionally or alternatively, in some embodiments, the recipient is a post-transplant recipient.

The following description is presented to enable a person of ordinary skill in the art to make and use various embodiments. Descriptions of specific devices, techniques, and applications are provided only as examples. These examples are being provided solely to add context and aid in the understanding of the described examples. It will thus be apparent to a person of ordinary skill in the art that the described examples may be practiced without some or all of the specific details. Other applications are possible, such that the following examples should not be taken as limiting. Various modifications in the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to a person of ordinary skill in the art, that one or more aspects and/or features described or referenced herein may be practiced without some or all of these specific details. In other instances, well-known process steps and/or structures have not been described in detail in order to not obscure some of the aspects and/or features described or referenced herein.

In the following description of examples, reference is made to the accompanying drawings which form a part hereof, and in which, by way of illustration, specific examples are shown that can be practiced. It is to be understood that other examples can be used and structural changes can be made without departing from the scope of the disclosed examples.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combination of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

### Exemplary User Interfaces for an Exemplary Pre- or Post-Transplant Recipient Self-Care Application

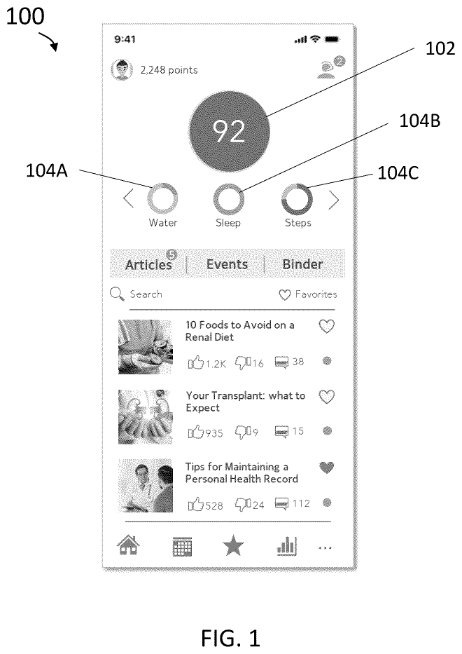

FIG. 1 illustrates an exemplary view of a user interface associated with a home screen of an app, according to embodiments of the disclosure. The home screen 100 may be a user interface displayed on the display of a device (e.g., a mobile phone, a tablet, a laptop computer, etc.). The home screen 100 can include one or more first graphical user interface (GUI) images 102 and one or more second GUI images 104. Although the figure identifies two sets of GUI images, embodiments of the disclosure may include any number and type of GUI objects (e.g., buttons, sliders, etc.).

The first GUI image 102 may be a graphic representative of one or more scores, such as a pre- or post-transplant recipient's overall score (discussed in more detail below). The one or more scores may be provided to the pre- or post-transplant recipient and/or a medical care provider by displaying the one or more scores on the display of an electronic device. In some embodiments, the score may be a single numerical value. The score may be masked or unmasked. A masked score may be one where the underlying data used to determine the score may not be accessible to the user, while the underlying data may be accessible when the score is unmasked.

The first GUI image 102 may include a color that corresponds to the displayed overall score. The color may represent a level of the pre- or post-transplant recipient's performance in meeting one or more health (physical or physiological) metric targets, behavioral metric targets, episode targets, or a combination thereof, over a time window (e.g., the last four days, the last week, the last month, etc.). For example, green may indicate good performance, yellow may indicate mediocre performance, and red may indicate poor performance. Embodiments of the disclosure may include other colors such as different shades of green, orange, etc. Additionally or alternatively, the GUI 100 may display a message associated with the overall score. For example, the GUI 100 may display an overall score of 100, a message associated with the overall score (e.g., “YES! You are doing great taking care of yourself!”), or both. As another example, the GUI may display an overall score of 0, a message associated with the overall score (e.g., “NO! You need to do a better job taking care of yourself!”), or both.

The second GUI images 104 may be graphics representative of the pre- or post-transplant recipient's progress towards meeting one or more metric targets. For example, the second GUI image 104A may indicate the pre- or post-transplant recipient's current water consumption, the second GUI image 104B may indicate the pre- or post-transplant recipient's sleep (total hours slept, hours of uninterrupted sleep, hours of deep sleep and/or light sleep, hours napped, and so forth), and the second GUI image 104C may indicate the steps the pre- or post-transplant recipient has taken during a particular time window, e.g., the current day. In some embodiments, the second GUI images 104 may indicate the pre- or post-transplant recipient's progress over a certain time period (e.g., the current day, the current week, etc.). In some embodiments, the second GUI images 104 may indicate a certain percentage of progress, such as how close the pre- or post-transplant recipient is towards meeting the one or more metric targets.

The home screen 100 may also display other information such as suggested articles for the pre- or post-transplant recipient to read and the number of points the pre- or post-transplant recipient has accrued, as shown in the upper left corner of the figure. The points may be related to daily goals and rewards (e.g., a login reward for logging into the app, a milestone reward for reaching milestones, etc.). In some embodiments, the pre- or post-transplant recipient may be rewarded points based on an overall score, a daily score, one or more health metric scores, one or more behavioral metric scores, one or more episode scores, or a combination thereof.

In some embodiments, the app may allow the pre- or post-transplant recipient to schedule an episode (e.g., an office visit with a medical care provider, a blood draw appointment, a biopsy of the transplanted organ, and so forth), view or monitor test results (e.g., blood test results, biopsy test results, and so forth), communicate with other pre- or post-transplant recipients (e.g., via a chat board, private messaging, and so forth), communicate with a medical care provider (e.g., to participate in decisions about the care of the pre- or post-transplant recipient, to schedule a medical or testing appointment, and so forth), etc. An episode may be an event that does not occur daily. Exemplary non-limiting episodes may include scheduling a blood draw, receiving a blood draw, scheduling a medical office visit, attending a scheduled medical office visit, completing a medical care related event, etc. The information from the test results may be used to compute one or more scores, for example, and may be included in the computation of the daily score or an overall score.

In some embodiments, only eligible pre- or post-transplant recipients may be able to earn points. Points may be allocated to an eligible pre- or post-transplant recipient using a ruleset, by an administrator, or both. A ruleset may cause one or more actions (e.g., delivering points and/or badges) to occur based on data, states, actions, calculations, or a combination thereof. The rulesets may be defined by and/or the actions may be performed by any type of pre- or post-transplant recipient including, but not limited to, an end transplant recipient, another transplant recipient (e.g., a transplant buddy), and a system administrator. In some embodiments, a state may be an event such as logging into the system (e.g., the app), reaching a metric target, posting a message to the chat board or private messaging, delivering points, etc.

The points delivered may differ depending on the milestone. Table 1 illustrates exemplary milestones and rewards.

TABLE 1
Exemplary milestones and rewards

| Milestone | Reward(s) |
|---|---|
| Reach water consumption target for seven consecutive days | Badge |
| Reach water consumption target for 30 consecutive days | Badge |
| | 10 points |
| Reach medication adherence target of 100% for seven | 10 points |
| consecutive days | |
| Reach medication adherence target of 100% for 30 | 50 points |
| consecutive days | |
| Reach mood logging target for seven consecutive days | Badge |
| Reach weight logging target for 14 consecutive days | Badge |
| Reach blood pressure logging target for 14 consecutive days | Badge |
| Reach urine output logging target for seven consecutive | Badge |
| days | |
| Reach 100% compliance with all goals for 30 days | 50 points |

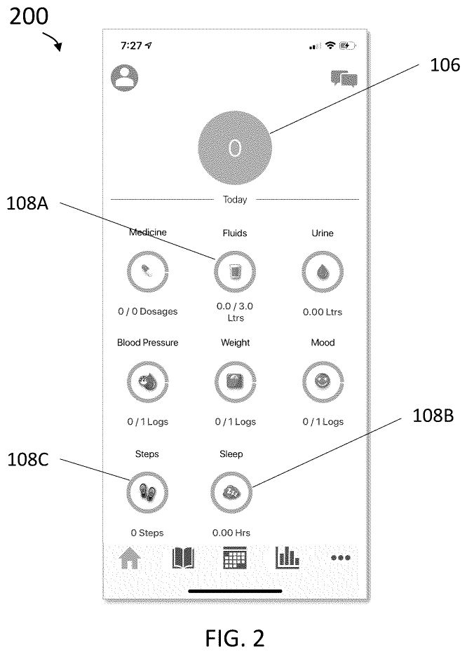

Embodiments of the disclosure may include multiple user interfaces for displaying different information. FIG. 2 illustrates an exemplary view of a user interface associated with a tracking screen of an app, according to embodiments of the disclosure. The tracking screen 200 may be used to provide information regarding one or more health (physical or physiological) metrics, behavioral metrics, episodes, or a combination thereof. The tracking screen 200 may also provide the pre- or post-transplant recipient's performance with respect to the health metric targets, behavioral metric targets, episode targets, or a combination thereof. The tracking screen 200 may be a user interface displayed on the display of a device (e.g., a mobile phone, a tablet, a laptop computer, etc.). The tracking screen 200 may include one or more third GUI images 106 and one or more fourth GUI images 108. Although the figure identifies two sets of GUI images, embodiments of the disclosure may include any number and type of GUI objects.

The third GUI image 106 may be a graphic representative of a score, such as a pre- or post-transplant recipient's daily score (discussed in more detail below). The third GUI image 106 may include a color that corresponds to the displayed daily score. The color may represent a level of the pre- or post-transplant recipient's performance in meeting one or more health metric targets, behavioral metric targets, episode targets, or a combination thereof, for the current day. For example, green may indicate good performance, yellow may indicate mediocre performance, and red may indicate poor performance. Embodiments of the disclosure may include other colors such as different shades of green, orange, etc. Additionally or alternatively, the GUI 200 may display a message associated with the daily score. For example, the GUI 200 may display a daily score of 100, a message associated with the daily score (e.g., “YES! You are doing great taking care of yourself!”), or both. As another example, the GUI 200 may display a daily score of 0, a message associated with the daily score (e.g., “NO! You need to do a better job taking care of yourself!”), or both.

The fourth GUI images 108 may be graphics representative of the pre- or post-transplant recipient's progress towards metric targets. For example, the fourth GUI image 108A may indicate the number of liters of water the pre- or post-transplant recipient has consumed during a particular time window, e.g., the current day, the fourth GUI image 108B may indicate the number of hours the pre- or post-transplant recipient had slept during a particular time window, e.g., the preceding night, and the fourth GUI image 108C may indicate the number of steps the pre- or post-transplant recipient has taken during a particular time window, e.g., the current day. In some embodiments, one or more fourth GUI images 108 may also provide numerical values regarding one or more of the health (physical or physiological) metrics, the behavioral metrics, episodes, the health (physical or physiological) metric targets, the behavioral metric targets, or the episode targets. For example, the fourth GUI image 108A may include text that shows the pre- or post-transplant recipient has consumed 0.0 liters of water and has a water consumption target of 3.0 liters.

In some embodiments, the tracking screen 200 may reset after a period of time (e.g., each day). After resetting, the transplant recipient's progress and daily score may be set to pre-determined values (e.g., zero).

### Exemplary Methods for Determining One or More Transplant Recipient Scores

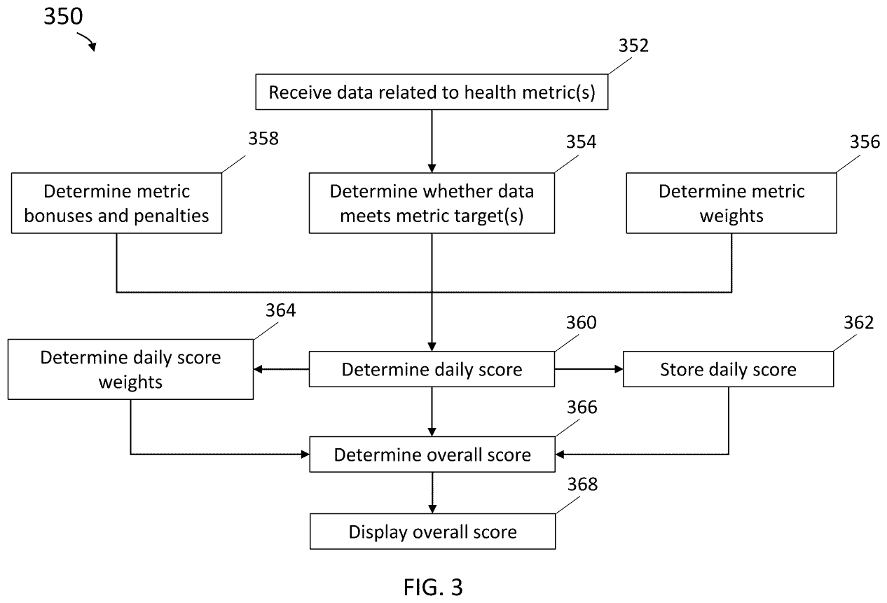

The system may determine one or more pre- or post-transplant recipient scores, and the pre- or post-transplant recipient score(s) may be used as quantifier(s) to evaluate the pre- or post-transplant recipient's overall activities and behaviors. FIG. 3 illustrates an exemplary flowchart for determining one or more pre- or post-transplant recipient scores, according to embodiments of the disclosure. Process 350 may begin with step 352. In step 352, the system may receive data related to one or more health and/or behavioral metrics.

In some embodiments, the metrics received in step 352 may be health (physical and physiological) metrics. Exemplary health metrics may include, but are not limited to, water consumption, calorie consumption, urination information, blood pressure, body weight, number of steps taken, number of hours slept, blood oxygen levels, blood glucose levels, pulse, and the like. In some embodiments, the data may include a numerical value that represents a health metric. For example, a numerical value of eight may be used to represent the number of hours slept.

The system may use any method for receiving the data related to the health metrics, such as receiving the transplant recipient's input (a manual input), taking one or more measurements using one or more devices, etc. The one or more devices may include, but are not limited to, biosensors, such as for measuring blood pressure, pulse, blood oxygen, etc., a thermometer for measuring temperature, third-party apps, Bluetooth devices, wearable electronics, etc. In some embodiments, the system may be configured to integrate data from one or more devices in a seamless manner.

For example, the pre- or post-transplant recipient may use the user interface 200 shown in FIG. 2 to input his or her hours slept during a preceding period of time. The device used for taking measurement(s) may be the same device that runs the app, or may be a measurement device coupled to the device that runs the app. In some embodiments, the device used for taking the measurement(s) may also be providing the daily score, the overall score, or both to the pre- or post-transplant recipient. In some embodiments, the measurement device may use one or more sensors included in the measurement device for taking measurement(s). Alternatively, the measurement device may be a remote device, where the device that runs the app may receive the data from the remote device.

Additionally or alternatively, the metrics received in step 352 may be one or more behavioral metrics. Exemplary behavioral metrics may include, but are not limited to, medication adherence, mood levels, compliance with episodes such as scheduled medical tests (such as blood draws, transplant biopsies, e.g., scheduled blood draws required for recommended post-transplant health surveillance, scheduled blood draws required to test transplant donor-recipient compatibility in case of pre-transplantation, e.g., Human Leukocyte Antigen (HLA) typing) or scheduled medical office visits, results (e.g., from blood, urine, saliva and other tests), and the like. Additional behavioral metrics may include time of exposure to ultraviolet (UV) radiation (sun exposure) with or without the application of protective gear or sun protective agents capable of absorbing UV rays. Based on the type of transplant that the post-transplant recipient has received or the type of transplant that the pre-transplant recipient is in need of, additional metrics may be included. Such additional metrics may include, but are not limited to, results from one or more electrocardiograms, e.g., in case of a heart pre- or post-transplant recipient; results from pulmonary function tests, e.g., a lung volume test, in case of a lung pre- or post-transplant recipient, adherence to special diet restrictions, e.g., limited intake of salt, sugar, cholesterol, saturated fatty acids in case of a kidney, heart, lung, liver, or pancreas pre- or post-transplant recipient, and so forth.

In some embodiments, the data may include a numerical value that represents a behavioral metric. For example, a numerical value of one may represent a low/pessimistic mood level.

In some embodiments, the system may receive the behavioral metrics data by receiving manual input from a pre- or post-transplant recipient, or receiving data from one or more devices or sources. For example, one source of data may be data from one or more electronic medical records (EMRs) to provide information about the pre- or post-transplant recipient's compliance with scheduled blood drawings or information about test results. For example, episode information may be obtained from a pre- or post-transplant recipient's EMR. Other exemplary non-limiting sources may include one or more medical care providers, one or more medication providers (pharmacies), etc. In some embodiments, certain information, such as an episode (a scheduled medical office visit or an emergency room visit) may not be available on the EMR system, but the transplant recipient may provide such information as manual input.

In step 354, the system may determine whether the data related to the health metrics and/or behavioral metrics meet one or more metric targets. In some embodiments, a metric target may be a threshold numerical value that represents a goal the pre- or post-transplant recipient is trying to reach and/or exceed. The metric target may have the same unit as the associated health metric and/or behavioral metric. One example metric target may be associated with the pre- or post-transplant recipient's body weight. The pre- or post-transplant recipient's body weight may be 243 lbs. at 9:00 am, 245 lbs. at 11:00 am, and 243 lbs. at 6:00 pm in a given day. The metric target may be 243 lbs. or less, which may have the same units as the associated health metric. Another example metric target may be associated with the transplant recipient's daily mood, e.g., as expressed as a numerical value out of a maximum such as 5 out of 10, with 1 being in a low, pessimistic, and/or depressed mood, and 10 being in high, optimistic spirits, or vice versa. The transplant recipient's daily mood may be 5 on Monday, 3 on Tuesday, 7 on Wednesday, etc.

In some embodiments, at least one metric target may be associated with an episode (e.g., the number of pre- or post-transplant recipient's inputs or measurements). Returning to the previous example, the metric target may be three measurements of the pre- or post-transplant recipient's weight per day. Since the pre- or post-transplant recipient provided input for three measurements (243 lbs. measured at 9:00 am, 245 lbs. measured at 11:00 am, and 243 lbs. measured at 6:00 pm) in a given day, the goal associated with the metric target may have been met.

An episode may or may not be associated with an episode target. An episode target may set one or more conditions to be met. One example condition may be reaching a state of completion. Another example condition may be reaching a state of completion within a predetermined time window. Exemplary episode targets may include, but are not limited to, completing an ordered blood or urine test, a biopsy, attending a scheduled medical office visit, etc. In some embodiments, a first episode target may be predicated on another target (e.g., a second episode target). For example, a first episode target may be a clinician ordering a test for a pre- or post-transplant recipient, and a second episode target may be the pre- or post-transplant recipient scheduling a blood draw (test) to complete the test order.

The system may store and access the metric targets in a file, such as a metric targets profile. The metric targets may be values personalized to the pre- or post-transplant recipient and may be determined by a clinician, for example. In some embodiments, the system may be capable of changing the metric targets. The change in metric targets may be dynamic or may be pre-set. For example, the system may change a metric target (e.g., water consumption target) based on historical self-care status, such as after the pre- or post-transplant recipient has consistently met a health metric (e.g., water consumption) for a period of time (e.g., three days). As another example, the system may change a metric target (e.g., medication dosage target) after a pre-set factor (e.g., the third day after a surgery). In some embodiments, a metric target may be determined based on the activities and behaviors of similar pre- or post-transplant recipients. The other pre- or post-transplant recipients may be similar with respect to one or more factors, such as demographics, type of transplant, e.g., kidney transplant, heart transplant, liver transplant, lung transplant, pancreas transplant, cornea transplant, cellular transplant, and so forth.

The determination in step 354 may lead to the system determining a raw score representative of how well the pre- or post-transplant recipient performed (e.g., adherence) for a given metric. In some embodiments, the system may assign one or more metrics with a raw score. The raw score may be a numerical value indicative of the pre- or post-transplant recipient's progress towards meeting a metric target. For example, a raw score of a maximum value (e.g., 100) may mean the pre- or post-transplant recipient has met the metric target. The difference between the maximum value and the raw score may indicate how close the pre- or post-transplant recipient is to meeting the metric target. In some embodiments, the raw score may be a value between 0-100. For example, the pre- or post-transplant recipient's medication adherence may have a raw score of 80, water consumption may have a raw score of 100, blood pressure may have a raw score of 0, urine production may have a raw score of 100, sleep tracking may have a raw score of 100, and mood tracking may have a raw score of 80. A higher raw score may indicate better performance relative to the respective metric target, for example.

In some embodiments, at least one raw score may be a binary value (e.g., 0 or 1) to indicate whether or not a measurement was taken for a given metric.

In some embodiments, the system may not use data related to one or more metrics in determining the daily score (in step 360), the overall score (in step 366), or both. The system may not determine a raw score for a metric not used for determining the daily score and/or overall score.

In step 356, the system may determine metric weights associated with the health metrics, behavioral metric, or episodes. Different pre- or post-transplant recipient activities may have different levels of importance, so the metric weights may be used to reflect the different levels of importance. In some embodiments, each metric weight may have a value between 0-1. For example, the pre- or post-transplant recipient's medication adherence may have a weight of 1.00, water consumption may have a weight of 0.96, blood pressure may have a weight of 0.8, urine production may have a weight of 0.6, sleep tracking may have a weight of 0.5, and mood tracking may have a weight of 0.4. In some embodiments, the metric weights may be pre-determined. In some embodiments, the metric weights may be dynamically adjusted. For example, the metric weights may be based on historical behaviors, such as past overall scores. In some embodiments, the metric weights may be time-based, decaying in value based on how current the information is.

In some embodiments, one or more episodes (e.g., each episode) may have a weight associated with it. Health activities may have varying levels of importance, where a weighting multiplier may reflect a level of importance. The weighting multiplier may, for example, be a numerical value between 0-1. For example, a first episode (completing a blood draw or test) may have a higher level of importance than a second episode (attending a medical care provider's appointment). These different levels of importance may be reflected in the one or more scores by assigning a weight to the first episode that is higher than the weight assigned to the second episode.

The system may store the metric weights in a file, such as a metric weights profile. The metric weights may be values personalized to the pre- or post-transplant recipient and may be determined by a medical care provider, for example. In some embodiments, the system may be capable of changing the metric weights. The change in metric weights may be dynamic (e.g., based on historical self-care status) or may be pre-set (e.g., based on a pre-set factor).

In step 358, the system may determine metric bonuses and penalties associated with the health metrics, behavioral metrics, or episodes. The system may associate a bonus or a penalty with a metric (e.g., at least one metric) when the pre- or post-transplant recipient's activity fails, reaches, or exceeds a certain metric target. The bonus or penalty may be used to account for excesses and shortfalls. For example, drinking too much water (e.g., drinking a certain number of ounces more than the target ounces) or not drinking water evenly throughout the day (e.g., drinking all the ounces of water in a short period of time) may be more detrimental than not drinking enough (i.e., the number of ounces that the pre- or post-transplant recipient drank is less than the target number of ounces). In such instance, the system may associate a penalty with the water consumption health metric. As another example, if a medical care provider orders a blood or urine test, but the pre- or post-transplant recipient does not schedule a blood draw within a certain time window, a penalty may be associated with the episode; such penalty may be reflected in the one or more scores.

Although the figure illustrates step 354, step 356, and step 358 as occurring at the same time, embodiments of the disclosure may include these steps occurring at different times. For example, the system may perform step 356 before step 354, or the system may perform step 356 first, followed by steps 354, and 358 next.

In step 360, the system may receive the information from step 354 (e.g., health metrics, behavioral metrics, episodes, etc.), step 356 (e.g., metric weights), and/or step 358 (e.g., metric bonuses and penalties) to determine a daily score. The system may multiply each value representative of the metrics by the associated metric weight and metric bonus/penalty to determine the daily score. The daily score may comprise a daily metric score and a daily episode score. The daily metric score may be a score based on the pre- or post-transplant recipient's health metrics, behavioral metrics, or both. The daily episode score may be a score based on an episode target, such as reaching a state of completion (of the episode) and within a predetermined time window. In some embodiments, the score may be calculated using data from all activities and behaviors tracked by the app.

In some embodiments, the system may multiply each value representative of the metrics by the associated metric weight and metric bonus/penalty to determine each metric score. The metric scores may be used to determine the number of points the pre- or post-transplant recipient has accrued. In some embodiments, the metric scores may not be included in the determining of the daily score, the overall score, or both.

In some embodiments, the daily score may be determined and/or updated when the system receives data related to the metrics (e.g., step 352). Alternatively, the daily score may be calculated at pre-determined times, such as at the end of the day when the system has received all data related to the metrics for the day.

Table 2 illustrates exemplary data related to health metrics, metric targets, raw scores for the health metrics, and metric weights. Although the table illustrates health metrics and health metric targets, embodiments of the disclosure are not limited to such and include other metrics/metric targets such as behavioral metrics/metric targets and episodes/episode targets.

TABLE 2
Exemplary health metrics, data, metric targets, raw scores, and
metric weights

| | Type | | | Raw Score | |
|---|---|---|---|---|---|
| | of Health | | Metric | for Health | Metric |
| Health Metric | Metric | Data | Target | Metric | Weight |
| Medication Adherence | Event | 18 | 16 | 100 | 0.9 |
| Water Consumption | Target | 3 | 2 | 100 | 0.7 |
| Blood Pressure | Event | 1 | 1 | 100 | 0.4 |
| Urine Production | Event | 1 | 1 | 100 | 0.4 |
| Step Tracking | Target | 500 | 1000 | 50 | 0.3 |
| Sleep Tracking | Target | 7.5 | 7 | 100 | 0.3 |
| Mood Tracking | Event | 4 | 3 | 100 | 0.3 |

In the example shown in Table 2, the app may receive and use data for seven health metrics. Of the seven health metrics, four health metrics are event-type health metrics that evaluate the number of events related to the health metric that occur (e.g., taking medication 16 times). Three health metrics are target-type health metrics that evaluate an amount related to the health metric (e.g., water consumption of 2.0 liters). The pre- or post-transplant recipient met the metric targets for six of the seven health metrics, and as a result, received a 100 raw score for the six health metrics (e.g., all health metrics except step tracking).

In some embodiments, the raw score may be a number representative of a percentage. For example, the number of steps taken by the pre- or post-transplant recipient may be equal to half of the metric target, and thus, the raw score for step tracking can be 50, which represents 50%. As shown in the table, at least two of the metrics have different metric weights, signifying importance to the pre- or post-transplant recipient's self-care. For example, medication adherence may be considered the most important metric, and thus, it may have the highest metric weight of 0.9. Step tracking, sleep tracking, and mood tracking may be considered the least important metric (among the seven health metrics), and thus, these metrics may have the lowest metric weight of 0.3. From the metrics, data, metric targets, raw scores, and metric weights, the system may determine the daily score is 95.

In step 362, the system may store the daily score determined in step 360. The daily score may be stored in any storage media, such as memory included in the device, on a server, on a remote device, or on the cloud.

The system may also determine the daily score weights in step 364. In some embodiments, the system may apply a daily score weight to one or more daily scores. The daily score weights may vary and may be based on the number of days passed since the current day, for example. In some embodiments, the daily score weight may be a value between 0 and 1. For example, the daily score weight for today may be 1.0, the daily score weight for yesterday may be 0.8, the daily score weight for two days ago may be 0.5, and the daily score weight for three days ago may be 0.1. In this manner, a higher daily score weight may be assigned to more recent daily scores.

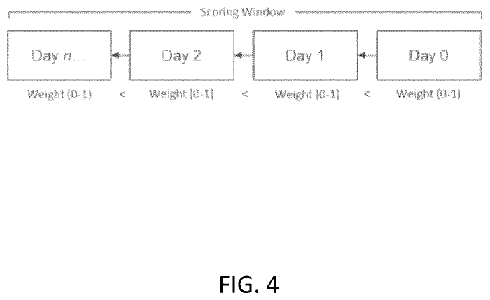

In step 366, the system may determine the overall score. The overall score may be determined based on the daily score weights and the daily scores within a scoring time window, as shown in FIG. 4. For example, the pre- or post-transplant recipient's daily score may be 98 on Monday, 82 on Tuesday, 84 on Wednesday, and 92 on Thursday. The weighted average daily score for a time window of four days may be equal to the weighted average of 98, 82, 84, and 92.

As another example, the scoring time window may be five days. As shown in Table 3, day 0 may have a daily score of 95, day 1 may have a daily score of 85, day 2 may have a daily score of 90, day 3 may have a daily score of 82, and day 4 may have a daily score of 92. Day 0 may be today, and thus, may have the highest weight of 1.0. Day 1 may have a weight of 0.8, day 2 may have a weight of 0.5, day 3 may have a weight of 0.2, and day 4 may have a weight of 0.1. Thus, the overall score may be equal to 90.

TABLE 3
Exemplary daily scores and daily score weights

| Day | Daily Score | Weight |
|---|---|---|
| 0 | 95 | 1.0 |
| 1 | 85 | 0.8 |
| 2 | 90 | 0.5 |
| 3 | 82 | 0.2 |
| 4 | 92 | 0.1 |

In some embodiments, the overall score may be a value from 0-100. A higher score may indicate that the pre- or post-transplant recipient is doing a better job at taking care of himself or herself, compared to a lower score, for example.

Returning back to FIG. 3, the system may display the overall score to the pre- or post-transplant recipient (step 368). For example, as shown in FIG. 1, the GUI 100 may display an overall score of 92 in GUI 100. Additionally or alternatively, the system may display other information, such as the change in overall score, metric targets that the transplant recipient has met, suggestions for improving self-care, etc.

In some embodiments, the pre- or post-transplant recipient's daily score, overall score, or both may be compared to those of other similar pre- or post-transplant recipients. The other pre- or post-transplant recipients may be similar with respect to one or more factors, such as demographics, type of transplant, e.g., kidney transplant, heart transplant, liver transplant, lung transplant, pancreas transplant, cornea transplant, cellular transplant, and so forth. The pre- or post-transplant recipient's relative score(s) may be displayed on a GUI, for example.

In some embodiments, the information (e.g., metrics, daily score(s), overall scores(s), etc.) determined at any one of the steps may be accessed by another user (e.g., a medical care provider) to help assess the pre- or post-transplant recipient's progress (e.g., in recovering from an illness, surgery, etc.). For example, the system and methods disclosed herein may provide a recommended personalized treatment regimen for post-transplant recipients (e.g., recipients of an allogeneic organ transplant or allogeneic cells). In some embodiments, the metric targets may be set based on the allogeneic organ transplant or allogeneic cells for the transplant recipient. Exemplary organs that may be transplanted include, for example, kidney, heart, lung, liver, pancreas, and cornea. Exemplary allogeneic cells that may be administered to the recipient include, but are not limited to, blood cells, stem cells, cardiomyocytes, neurons, lymphocytes, NK cells, NKT cells, T reg cells, macrophages, dendritic cells, and pancreatic islet cells. In some embodiments, the allogeneic cells are allogeneic blood cells. Allogeneic blood cells may include hematopoietic stem cells (i.e., HSCs), T cells, B cells, and CAR T cells, NK cells, NKT cells, and TILs. The specific tracked metrics will depend on the particular type of transplant or allogeneic cells transferred to the recipient.

### Exemplary System for an Exemplary Self-Care Application

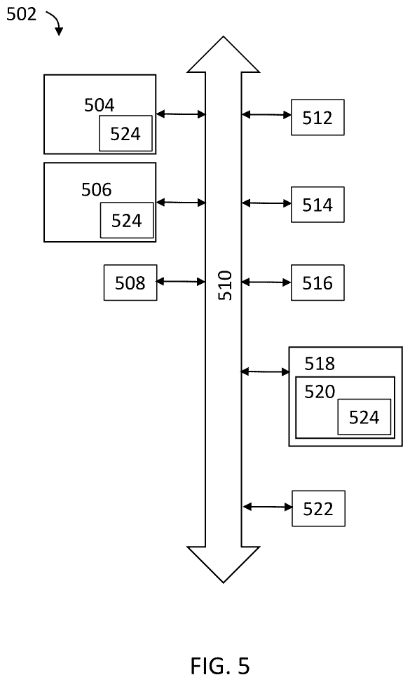

The system and methods (self-care app) discussed above may be implemented by a device. FIG. 5 illustrates an exemplary device that implements the above disclosed system, according to embodiments of the disclosure. The device 502 may be a portable electronic device, such as a cellular phone, a tablet computer, a laptop computer, or a wearable device. The device 502 can include a processor 504 (e.g., a central processing unit (CPU), a graphics processing unit (GPU), or both), a main memory 506 (e.g., read-only memory (ROM), flash memory, dynamic random access memory (DRAM) such as synchronous DRAM (SDRAM) or Rambus DRAM (RDRAM), etc.), and a static memory 508 (e.g., flash memory, static random access memory (SRAM), etc.), which can communicate with each other via a bus 510.

The device 502 may also include a display 512, an input/output device 514 (e.g., a touch screen), a transceiver 516, and storage 518. Storage 518 includes a machine-readable medium 520 on which is stored one or more sets of instructions 524 (e.g., software) embodying any one or more of the methodologies or functions described herein. The software may also reside, completely or at least partially, within the main memory 506 and/or within the processor 504 during execution thereof by the computer 502, the main memory 506, and the processor 504 also constituting machine-readable media. The software may further be transmitted or received over a network via a network interface device 522.

While the machine-readable medium 520 is shown in an exemplary embodiment to be a single medium, the term “machine-readable medium” should be taken to include a single medium or multiple media (e.g., a centralized or distributed database and/or associated caches and servers) that store the one or more sets of instructions. The term “machine-readable medium” shall also be taken to include any medium that is capable of storing, encoding, or carrying a set of instructions for execution by the machine and that causes the machine to perform any one or more of the methodologies of the present invention. The term “machine-readable medium” shall accordingly be taken to include, but not be limited to, solid-state memories, optical and magnetic media, and carrier wave signals.

The system and methods described herein (self-care app) and its data can be stored in storage 518, main memory 506, static memory 508, or a combination thereof. The self-care app may include a user interface for the home screen 100, the tracking screen 200, etc., as discussed throughout this disclosure. The display 512 may be used to present a user interface to the pre- or post-transplant recipient, and the input/output device 514 may be used to receive input (e.g., clicking on a graphic representative of a microblog) from the pre- or post-transplant recipient. The transceiver 516 may be configured to communicate with a network, for example.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A computer-implemented method for generating, outputting, and dynamically updating a metric progress aggregation insight interface in association with a pre- or post-transplant recipient, the method comprising:
receiving, at disparate timing intervals and via one or more metric collection mechanisms, data related to a plurality of disparate metric collection inputs, the one or more metric collection mechanisms comprising one or more sensors onboard a portable electronic device, one or more offboard sensors electronically coupled to the portable electronic device, a graphical user interface (GUI) of the portable electronic device, and one or more remote electronic medical record sources;
accessing, via a processor of the portable electronic device, a metric targets profile, wherein the metric targets profile includes a plurality of metric targets corresponding to at least the plurality of disparate metric collection inputs;
determining raw scores for the data related to the plurality of disparate metric collection inputs, wherein the raw scores are determined based on the metric targets profile;
accessing, via the processor of the portable electronic device, a metric weights profile, wherein the metric weights profile includes a plurality of metric weights corresponding to at least the plurality of disparate metric collection inputs;
determining, via relative importance determination logic, one or more aggregated scores based on the raw scores and the plurality of metric weights, wherein the relative importance determination logic accommodates the plurality of disparate metric collection inputs by weighting the raw scores based on relative importance to the pre- or post-transplant recipient;
generating a metric progress aggregation insight interface comprising the one or more aggregated scores;
outputting the metric progress aggregation insight interface to at least a display of the portable electronic device; and
dynamically updating the metric progress aggregation insight interface based on receiving additional data related to the plurality of disparate metric collection inputs.

2. The computer-implemented method of claim 1, wherein receiving the data related to the plurality of disparate metric collection inputs comprise receiving input of the data via the GUI of the portable electronic device.

3. The computer-implemented method of claim 1, wherein receiving the data related to the plurality of disparate metric collection inputs comprises taking one or more measurements using at least one of the one or more sensors onboard the portable electronic device.

4. The computer-implemented method of claim 1, wherein receiving the data related to the plurality of disparate metric collection inputs comprises taking one or more measurements using at least one of the one or more offboard sensors of a remote measurement device electronically coupled to the portable electronic device.

5. The computer-implemented method of claim 1, wherein at least one of the plurality of metric targets is associated with an episode.

6. The computer-implemented method of claim 1, further comprising:
dynamically changing the plurality of metric weights based on historical self-care status data associated with the pre- or post-transplant recipient.

7. The computer-implemented method of claim 1, wherein at least one of the raw scores is a binary value.

8. The computer-implemented method of claim 1, further comprising:
associating a bonus or a penalty with at least one of the plurality of disparate metric collection inputs.

9. The computer-implemented method of claim 1, wherein the one or more aggregated scores includes a daily score.

10. The computer-implemented method of claim 1, wherein dynamically determining, via the relative importance determination logic, the one or more aggregated scores includes determining an overall score, and wherein determining the overall score comprises:
determining daily score weights for a plurality of daily scores within a scoring time window, wherein the overall score is based on the daily score weights and the plurality of daily scores.

11. The computer-implemented method of claim 10, wherein the daily score weights vary and are based on a number of days since a current day.

12. The computer-implemented method of claim 1, wherein the one or more aggregated scores are included in a personalized treatment regimen for the pre- or post-transplant recipient, and the metric weights profile is based on an allogeneic organ transplant or allogeneic cells for the pre- or post-transplant recipient.

13. The computer-implemented method of claim 1, wherein:
(a) the plurality of disparate metric collection inputs is a plurality of health metrics and the plurality of metric targets is a plurality of health metric targets;
(b) the plurality of disparate metric collection inputs is a plurality of behavioral metrics and the plurality of metric targets is a plurality of behavioral metric targets; or
(c) the plurality of disparate metric collection inputs is a plurality of episodes and the plurality of metric targets is a plurality of episode targets.

14. The computer-implemented method of claim 13, wherein the plurality of health metrics comprises one or more of water consumption, calorie consumption, urination information, blood pressure, body weight, number of steps taken, number of hours slept, blood oxygen levels, blood glucose levels, or pulse.

15. The computer-implemented method of claim 13, wherein the plurality of behavioral metrics comprises one or more of medication adherence, mood levels, compliance with scheduled medical tests, compliance with medical office visits, or test results.

16. The computer-implemented method of claim 13, wherein the plurality of episodes comprises one or more of scheduling a blood or urine test, scheduling a medical office visit, attending a medical office visit, or receiving a blood or urine test.

17. The computer-implemented method of claim 1, further comprising:
dynamically changing at least one metric target of the plurality of metric targets to generate an updated plurality of metric targets; and
storing the updated plurality of metric targets in the metric targets profile, thereby initiating an update to the metric targets profile.

18. The computer-implemented method of claim 17, wherein dynamically changing at least one metric target of the plurality of metric targets comprises:
determining a similar pre- or post-transplant recipient set based on one or more similarity factors associated with the pre- or post-transplant recipient; and
dynamically changing the at least one metric target based on a determination of activities and behaviors associated with the similar pre- or post-transplant recipient set.

19. A non-transitory computer-readable storage medium for generating, outputting, and dynamically updating a metric progress aggregation insight interface in association with a pre- or post-transplant recipient, the non-transitory computer-readable storage medium storing one or more programs, the one or more programs comprising instructions, which when executed by one or more processors of a portable electronic device having a display, cause the portable electronic device to:
receive, at disparate timing intervals and via one or more metric collection mechanisms, data related to a plurality of disparate metric collection inputs, the one or more metric collection mechanisms comprising one or more sensors onboard a portable electronic device, one or more offboard sensors electronically coupled to the portable electronic device, a graphical user interface (GUI) of the portable electronic device, and one or more remote electronic medical record sources;
access a metric targets profile, wherein the metric targets profile includes a plurality of metric targets corresponding to at least the plurality of disparate metric collection inputs;
determine raw scores for the data related to the plurality of disparate metric collection inputs, wherein the raw scores are determined based on the metric targets profile;
access a metric weights profile, wherein the metric weights profile includes a plurality of metric weights corresponding to at least the plurality of disparate metric collection inputs;
determine, via relative importance determination logic, one or more aggregated scores based on the raw scores and the plurality of metric weights, wherein the relative importance determination logic accommodates the plurality of disparate metric collection inputs by weighting the raw scores based on relative importance to the pre- or post-transplant recipient;
generate a metric progress aggregation insight interface comprising the one or more aggregated scores;
output the metric progress aggregation insight interface to at least the display of the portable electronic device; and
dynamically update the metric progress aggregation insight interface based on receiving additional data related to the plurality of disparate metric collection inputs.

20. A portable electronic device for generating, outputting, and dynamically updating a metric progress aggregation insight interface in association with a pre- or post-transplant recipient, the portable electronic device comprising:
a display;
one or more processors;
memory; and
one or more programs, wherein the one or more programs are stored in the memory and are configured to be executed by the one or more processors, the one or more programs including instructions for:
receiving, at disparate timing intervals and via one or more metric collection mechanisms, data related to a plurality of disparate metric collection inputs, the one or more metric collection mechanisms comprising one or more sensors onboard a portable electronic device, one or more offboard sensors electronically coupled to the portable electronic device, a graphical user interface (GUI) of the portable electronic device, and one or more remote electronic medical record sources;
accessing, via the one or more processors, a metric targets profile, wherein the metric targets profile includes a plurality of metric targets corresponding to at least the plurality of disparate metric collection inputs;
determining raw scores for the data related to the plurality of disparate metric collection inputs, wherein the raw scores are determined based on the metric targets profile;
accessing, via the one or more processors, a metric weights profile, wherein the metric weights profile includes a plurality of metric weights corresponding to at least the plurality of disparate metric collection inputs;
determining, via relative importance determination logic, one or more aggregated scores based on the raw scores and the plurality of metric weights, wherein the relative importance determination logic accommodates the plurality of disparate metric collection inputs by weighting the raw scores based on relative importance to the pre- or post-transplant recipient;
generating a metric progress aggregation insight interface comprising the one or more aggregated scores;
outputting the metric progress aggregation insight interface to at least the display; and
dynamically updating the metric progress aggregation insight interface based on receiving additional data related to the plurality of disparate metric collection inputs.
